SEQUENCE LISTING (1 sequence)

SEQ ID NO: 1 - DNA, 2031 bp, Ebola virus
atgggtgttacaggaatattgcagttacctcgtgatcgattcaagaggacatcattctttctttgggtaattatccttttccaaagaacattttccatcccgcttggagttatccacaatagtacattacaggttagtgatgtcgacaaactagtttgtcgtgacaaactgtcatccacaaatcaattgagatcagttggactgaatctcgaggggaatggagtggcaactgacgtgccatctgtgactaaaagatggggcttcaggtccggtgtcccaccaaaggtggtcaattatgaagctggtgaatgggctgaaaactgctacaatcttgaaatcaaaaaacctgacgggagtgagtgtctaccagcagcgccagacgggattcggggcttcccccggtgccggtatgtgcacaaagtatcaggaacgggaccatgtgccggagactttgccttccacaaagagggtgctttcttcctgtatgatcgacttgcttccacagttatctaccgaggaacgactttcgctgaaggtgtcgttgcatttctgatactgccccaagctaagaaggacttcttcagctcacaccccttgagagagccggtcaatgcaacggaggacccgtcgagtggctattattctaccacaattagatatcaggctaccggttttggaactaatgagacagagtacttgttcgaggttgacaatttgacctacgtccaacttgaatcaagattcacaccacagtttctgctccagctgaatgagacaatatatgcaagtgggaagaggagcaacaccacgggaaaactaatttggaaggtcaaccccgaaattgatacaacaatcggggagtgggccttctgggaaactaaaaaaaacctcactagaaaaattcgcagtgaagagttgtctttcacagctgtatcaaacggacccaaaaacatcagtggtcagagtccggcgcgaacttcttccgacccagagaccaacacaacaaatgaagaccacaaaatcatggcttcagaaaattcctctgcaatggttcaagtgcacagtcaaggaaggaaagctgcagtgtcgcatctgacaacccttgccacaatctccacgagtcctcaacctcccacaaccaaaacaggtccggacaacagcacccataatacacccgtgtataaacttgacatctctgaggcaactcaagttggacaacatcaccgtagagcagacaacgacagcacagcctccgacactccccccgccacgaccgcagccggacccttaaaagcagagaacaccaacacgagtaagagcgctgactccctggacctcgccaccacgacaagcccccaaaactacagcgagactgctggcaacaacaacactcatcaccaagataccggagaagagagtgccagcagcgggaagctaggcttaattaccaatactattgctggagtagcaggactgatcacaggcgggagaaggactcgaagagaagtaattgtcaatgctcaacccaaatgcaaccccaatttacattactggactactcaggatgaaggtgctgcaatcggattggcctggataccatatttcgggccagcagccgaaggaatttacacagaggggctaatgcacaaccaagatggtttaatctgtgggttgaggcagctggccaacgaaacgactcaagctctccaactgttcctgagagccacaactgagctgcgaaccttttcaatcctcaaccgtaaggcaattgacttcctgctgcagcgatggggtggcacatgccacattttgggaccggactgctgtatcgaaccacatgattggaccaagaacataacagacaaaattgatcagattattcatgattttgttgataaaacccttccggaccagggggacaatgacaattggtggacaggatggagacaatggataccggcaggtattggagttacaggtgttataattgcagttatcgctttattctgtatatgcaaatttgtcttttag

(19) United States
(12) Patent Application Publication
(10) Pub. No.: US 2018/0353568 A1
(43) Pub. Date: Dec. 13, 2018

(54) USE OF TEICOPLANIN AGAINST EBOLA VIRUS

(71) Applicant: SUN YAT-SEN UNIVERSITY, Guangzhou (CN)

(72) Inventors: Ting PAN, Guangzhou (CN); Hui ZHANG, Guangzhou (CN); Nan ZHOU, Guangzhou (CN)

(73) Assignee: SUN YAT-SEN UNIVERSITY, Guangzhou (CN)

(21) Appl. No.: 15/568,151

(22) PCT Filed: Apr. 22, 2015
(86) PCT No.: PCT/CN2015/077207

§ 371 Date: Oct. 20, 2017

(51) Int. Cl. A61K 38/14; A61P 31/14

(52) U.S. Cl. CPC A61K 38/14; A61P 31/14

(57) ABSTRACT

The present invention discloses a use of teicoplanin against Ebola virus, and discloses a drug inhibiting an envelope protein GP that comprises the teicoplanin.

### TECHNICAL FIELD

The present invention relates to a new use of an antiviral drug, and more specifically, to a use of teicoplanin against Ebola virus.

### BACKGROUND

In 2014, an outbreak of Ebola virus in many countries of West Africa has killed tens of thousands of innocent people's lives. The outbreak is the most significant, the most severe and the most complicated one in the history of Ebola epidemic in recent 40 years. Before the outbreak of Ebola epidemic, the virus had ever been founded in 1967 in Africa, and then returned into the jungle quickly. Until now, the unimpressive Ebola virus is still swallowing the life of infected people at an extremely rare speed, almost without any resistance. The development of drugs seems so urgent, that even the drugs and vaccines that are unapproved yet have already gone into the battle. Ebola virus is a filamentous virus with envelope and single-stranded antisense RNA genome. The infection of the Ebola virus may lead to serious viral hemorrhagic fever in humans and non-human primates, and result in mortality up to 50-90%. But now, the word lacks effective vaccines and drugs against the Ebola virus. Therefore, as facing such a severe Ebola crisis, purposefully, designedly and systematically promoting the research and development work of drugs against the Ebola virus as soon as possible, and developing economical and convenient drugs without any side effects, have already been urgent tasks of preventing and fighting against the Ebola virus for the whole society nowadays, and of important significance.

### SUMMARY OF THE INVENTION

At present, there aren't any ready-made antiviral drugs against Ebola virus that can be used in the market.

The present invention provides a new use of a known drug, i.e. a use of teicoplanin against Ebola virus. The teicoplanin has a structural formula shown as formula I:

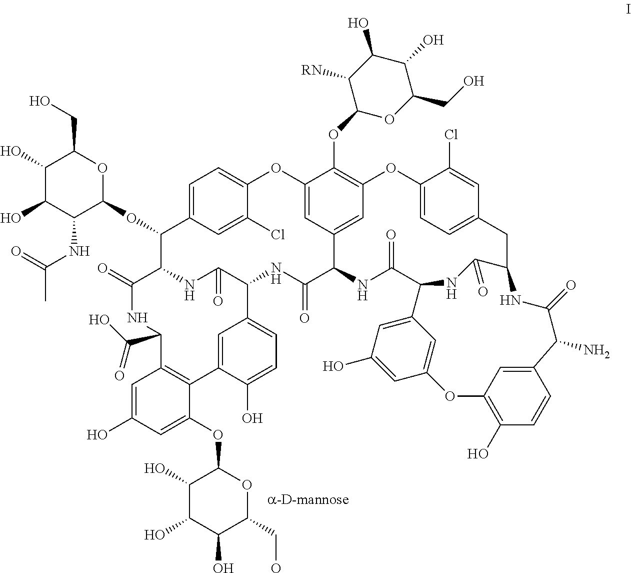

Wherein R is selected from a group consisting of

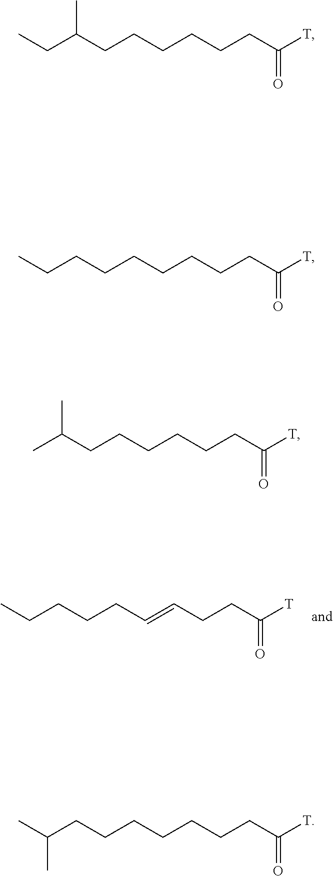

At present, the teicoplanin sold on the market actually is a mixture, and R is a side chain fatty acid.

The teicoplanin inhibits the envelope protein GP of Ebola virus, and especially can inhibit the Zaire type envelope protein that outbroke in 2014.

The teicoplanin inhibits the Ebola virus from entering into host cells.

More specifically, the use of teicoplanin in preparing a drug inhibiting the envelope protein GP is provided.

The advantages of the present invention are:

1. In the present invention, plasmids pHIV-luc, pCMV-deltaR8.2 and EBOV-GP were transfected into 293T cells, to package the pseudovirus that can express the envelope protein of Ebola virus, and then the 293T cells were infected by the pseudovirus to simulate a status of Ebola virus infecting the host cells.

2. In the present invention, that cell model packaged by the pseudovirus was applied to screen more than one thousand of the libraries that have been marketed and used, and thus a phenomenon that the antibiotic teicoplanin can effectively inhibit the Ebola virus infecting the 293T cells had been found. The antibiotic teicoplanin has been verified by many experiments to have a good antiviral function. The IC50 of teicoplanin was 200 nM.

3. Safety of the drug for human body had already been proved by clinical practice. Obvious efficacy of teicoplanin against Ebola virus has been confirmed presently. The drug can be directly used on the forefront of clinical therapy after being emergently recorded and approved by the State Drug Administration, avoiding the long developing period of new drugs, providing a powerful theoretical and practical basis for further development of drugs against Ebola virus, and is with great values of development and of important significance of promotion.

4. According to the research of the present invention, teicoplanin has obvious inhibitory effect on the envelope protein GP of Ebola virus, especially on the Zaire type envelope protein that outbroke in 2014 in West Africa.

### BRIEF DESCRIPTION OF THE DRAWINGS

FIG. 1 is the inhibitory effect of the antibiotic teicoplanin with different concentrations on the vesicular stomatitis virus envelope protein EBOV-G.

FIG. 2 is the inhibitory effect of the antibiotic teicoplanin with different concentrations on the vesicular stomatitis virus envelope G protein, VSV-G.

FIG. 3 is the CC50 of the antibiotic teicoplanin in 293T cells.

FIG. 4 is the IC50 of the antibiotic teicoplanin in 293T cells.

FIG. 5 is the inhibitory effect of the antibiotic teicoplanin with different concentrations in A549 cells of human lung cancer cell lines.

FIG. 6 is the inhibitory effect of the antibiotic teicoplanin with different concentrations in Hela cells of human cervical cancer cell lines.

FIG. 7 is the inhibitory effect of the antibiotic teicoplanin with different concentrations in human monocytic leukemia THP-1 cells.

FIG. 8 is the inhibitory effect of the antibiotic teicoplanin with different concentrations in human umbilical vein endothelial cells (HUVECs).

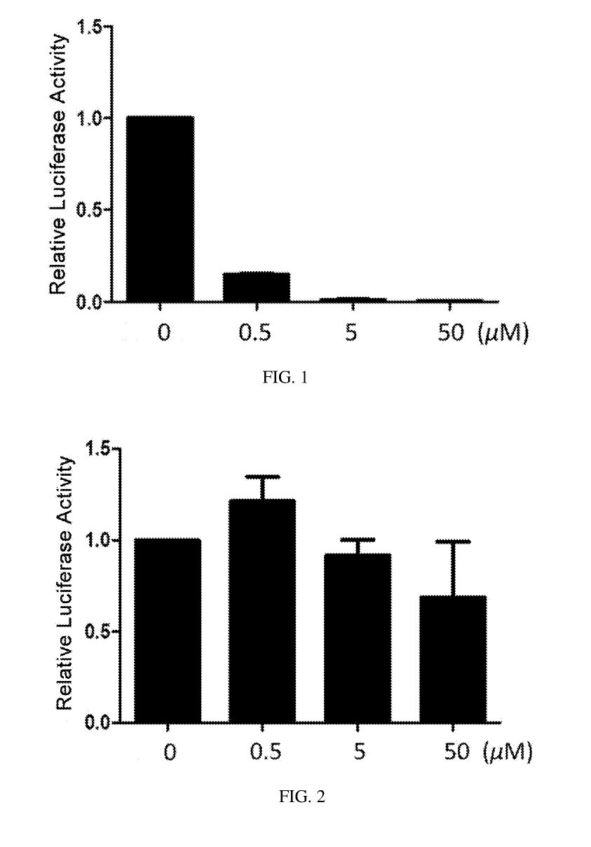

### DETAILED DESCRIPTION OF THE PREFERRED EMBODIMENT

The present invention is described in further details below by combining the accompanying drawings and specific embodiments. Unless specified, reagents and devices used in the present invention are conventional commercially available reagents and devices in the present technical field, and methods used in the present invention are conventional used methods.

The sequence of Zaire type envelope protein GP of Ebola virus is shown as SEQ ID NO. 1. Zaire EBOV-GP2014 shown in the accompanying drawings is the Zaire type envelope protein of Ebola virus.

### Embodiment 1: Inhibitory Effect of the Antibiotic Teicoplanin with Different Concentrations on the Envelope Protein GP of Ebola Virus

(1) Packaging virus: plasmids pHIV-luc, pCMV-deltaR8.2 and EBOV-GP were transfected into 293T cells (10 cm dish), and after 48 hours, the supernatant of the virus was collected for testing p24.

(2) Infecting: the 293T cells in a 96-well plate were infected by p24-normalized HIV-luc/EBOV-GP pseudotype virus containing 8 μg/mL polybrene, and the antibiotic teicoplanin with different concentrations were added at the same time.

(3) Changing medium: after 12 hours of infection, the medium was changed with fresh DNEM medium.

(4) Testing luciferase activity: after 48 hours of infection, each well was washed once with PBS, then 100 μL lysis buffer was added, a shaking was carried out for 30 min, and 10 μL of lysate was taken out for testing luciferase activity.

### Embodiment 2: Inhibitory Effect of the Antibiotic Teicoplanin with Different Concentrations on the Envelope G Protein of Vesicular Stomatitis Virus, VSV-G

(1) Packaging virus: plasmids pHIV-luc, pCMV-deltaR8.2 and VSV-G were transfected into 293T cells (10 cm dish), and after 48 hours, the supernatant of the virus was collected for testing p24.

(2) Infecting: the 293T cells in a 96-well plate were infected by p24-normalized HIV-luc/VSV-G pseudotype virus containing 8 μg/mL polybrene, and the antibiotic teicoplanin with different concentrations were added at the same time.

(3) Changing medium: after 12 hours of infection, the medium was changed with fresh DNEM medium.

(4) Testing luciferase activity: after 48 hours of infection, each well was washed once with PBS, and then 100 μL lysis buffer was added, a shaking was carried out for 30 min, and 10 μL of lysate was taken out for testing luciferase activity.

According to the results of the embodiment 1 and the embodiment 2, the antibiotic teicoplanin has specific effect on the envelope protein of Ebola virus, EBOV-GP, and can inhibit the entry of Ebola virus, and the antibiotic teicoplanin has no inhibitory effect on the envelope G protein of vesicular stomatitis virus, VSV-G.

### Embodiment 3: Toxicity CC50 Test

MTS (3-(4,5-dimethylthiazol-2-yl)-5(3-carboxymethoxyphenyl)-2-(4-sulfopheny)-2H-tetrazolium, inner salt) is a newly synthesized tetrazoles. It shares the same application principles with MTT, that is: being reduced into colored formazan products respectively by a plurality of dehydrogenases in mitochondria of living cells. The depth of color of the products is highly related to the number of living cells of some sensitive cell lines within a certain range. The number of living cells could be determined according to the measured absorbance value (OD value) at the wavelength of 490 nm. The larger the OD values, the greater the activity of cells, and indicating the lower the toxicity of the drug.

(1) Plating cells. 293T was formulated to single cell suspension by DMEM medium containing 10% fetal calf serum. The cells were plated into a 96-well plate as each well containing 1000 cells, with a volume of each well was 200 μL.

(2) After 24 hours of adherence culture, the antibiotic teicoplanin was added, 2 μL for each well, and the final concentration was 50 μM.

(3) After 48 hours of culturing, 20 pt of MTS solution was added into each well and an incubation was continued in an incubator for 2 to 4 hours.

(4) 490 nm was selected as the wavelength, the absorbance values were tested for each well on the enzyme-linked monitor, and the cell toxicity to 293T cells was observed.

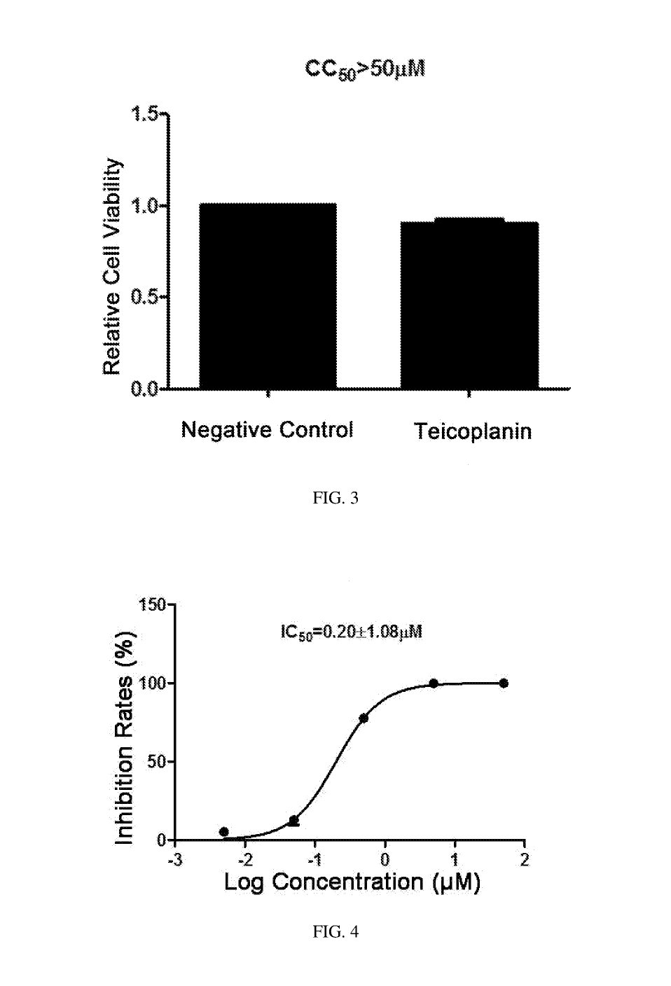

It can be observed from FIG. 3 that the toxicity of teicoplanin is low. Teicoplanin appears non-cytotoxic in the 293T cells as the concentration was 50 nM.

### Embodiment 4

(1) Packaging virus: plasmids pHIV-luc, pCMV-deltaR8.2 and EBOV-GP were transfected into 293T cells (10 cm dish), and after 48 hours, the supernatant of virus was collected for testing p24.

(2) Infecting: the 293T cells in a 96-well plate were infected by p24-normalized HIV-luc/EBOV-GP pseudotype virus containing 8 μg/mL polybrene, and the antibiotic teicoplanin with different concentrations were added at the same time. Final concentrations were 50 nM, 5 μM, 0.5 nM, 0.05 μM, 0.005 μM, and 0 μM, respectively.

(3) Changing medium: after 12 hours of infection, the medium was changed with fresh DNEM medium.

(4) Testing luciferase activity: after 48 hours of infection, each well was washed once with PBS, then 100 μL lysis buffer was added, a shaking was carried out for 30 min, and 10 μL of lysate was taken out for testing luciferase activity.

(5) According to the results tested, following IC50 curves as followed were drawn.

It can be observed from FIG. 4 that teicoplanin has good inhibitory effect on virus.

### Embodiment 5: Inhibitory Effect of the Antibiotic Teicoplanin with Different Concentrations in A549 Cells of Human Lung Cancer Cell Lines

(1) Packaging virus: plasmids pHIV-luc, pCMV-deltaR8.2 and Zaire EBOV-GP2014 were transfected into 293T cells (10 cm dish), and after 48 hours, the supernatant of virus was collected for testing p24.

Plasmids pHIV-luc, pCMV-deltaR8.2 and VSV-G were transfected into 293T cells (10 cm dish) at the same time, and after 48 hours, the supernatant of virus was collected for testing p24.

(2) Infecting: A549 cells of human lung cancer cell lines in a 96-well plate were infected by p24-normalized HIV-luc/Zaire EBOV-GP2014 or HIV-luc/VSV-G pseudotype virus containing 8 μg/mL polybrene, and the antibiotic teicoplanin with different concentrations were added at the same time.

(3) Changing medium: after 12 hours of infection, the medium was changed with fresh DNEM medium.

(4) Testing luciferase activity: after 48 hours of infection, each well was washed once with PBS, then 100 μL lysis buffer was added, a shaking was carried out for 30 min, and 10 μL of lysate was taken out for testing luciferase activity.

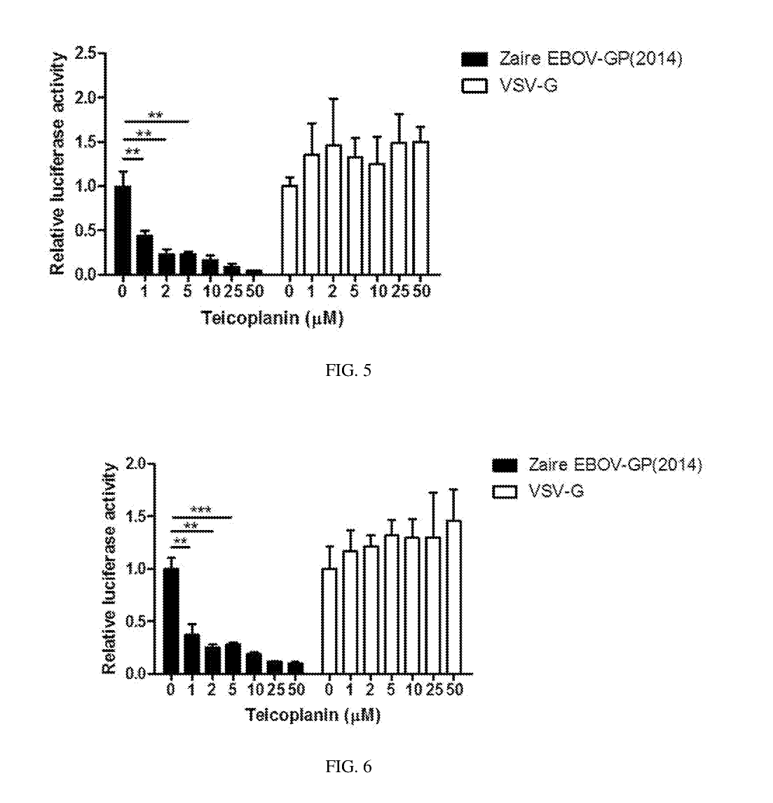

It can be observed from FIG. 5 that teicoplanin also has good inhibitory effect on virus in the A549 cells of the human lung cancer cell lines.

### Embodiment 6: Inhibitory Effect of the Antibiotic Teicoplanin with Different Concentrations in Hela Cells of Human Cervical Cancer Cell Lines

(1) Packaging virus: plasmids pHIV-luc, pCMV-deltaR8.2 and Zaire EBOV-GP2014 were transfected into 293T cells (10 cm dish), and after 48 hours, the supernatant of virus was collected for testing p24.

Plasmids pHIV-luc, pCMV-deltaR8.2 and VSV-G were transfected into 293T cells (10 cm dish) at the same time, and after 48 hours, the supernatant of virus was collected for testing p24.

(2) Infecting: Hela cells of human cervical cancer cell lines in a 96-well plate were infected with p24-normalized HIV-luc/Zaire EBOV-GP2014 or HIV-luc/VSV-G pseudotype virus containing 8 μg/mL polybrene, and the antibiotic teicoplanin with different concentrations were added at the same time.

(3) Changing medium: after 12 hours of infection, the medium was changed with fresh DNEM medium.

(4) Testing luciferase activity: after 48 hours of infection, each well was washed once with PBS, then 100 μL lysis buffer was added, a shaking was carried out for 30 min, and 10 μL of lysate was taken out for testing luciferase activity.

It can be observed from FIG. 6 that teicoplanin has also good inhibitory effect on virus in the Hela cells of the human cervical cancer cell lines.

### Embodiment 7: Inhibitory Effect of the Antibiotic Teicoplanin with Different Concentrations in Human Monocytic Leukemia THP-1 Cells

(1) Packaging virus: plasmids pHIV-luc, pCMV-deltaR8.2 and Zaire EBOV-GP2014 were transfected into 293T cells (10 cm dish), and after 48 hours, the supernatant of virus was collected for testing p24.

Plasmids pHIV-luc, pCMV-deltaR8.2 and VSV-G were transfected into 293T cells (10 cm dish) at the same time, and after 48 hours, the supernatant of virus was collected for testing p24.

(2) Infecting: human monocytic leukemia THP-1 cells in a 96-well plate were infected with p24-normalized HIV-luc/Zaire EBOV-GP2014 or HIV-luc/VSV-G pseudotype virus containing 8 μg/mL polybrene, and the antibiotic teicoplanin with different concentrations were added at the same time.

(3) Changing medium: after 12 hours of infection, the medium was changed with fresh DNEM medium.

(4) Testing luciferase activity: after 48 hours of infection, each well was washed once with PBS, then 100 μL lysis buffer was added, a shaking was carried out for 30 min, and 10 μL of lysate was taken out for testing luciferase activity.

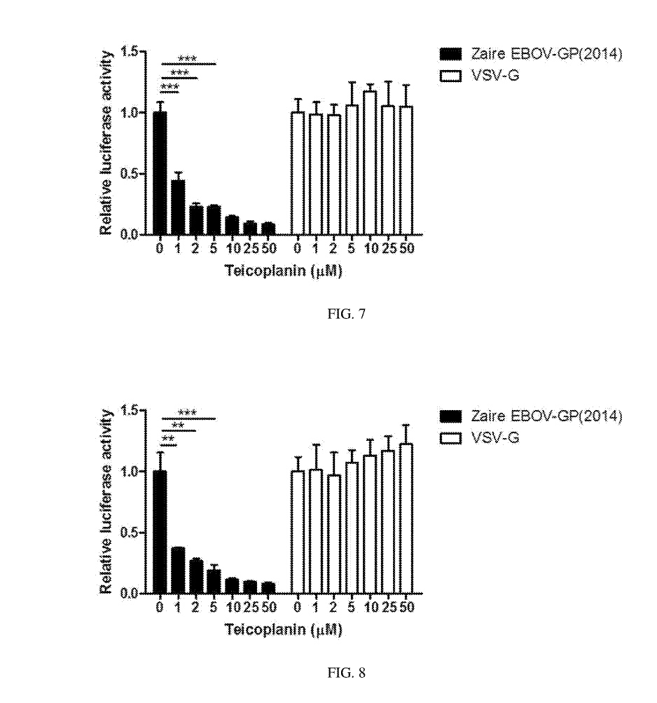

It can be observed from FIG. 7 that teicoplanin has also good inhibitory effect on virus in the human monocytic leukemia THP-1 cells.

### Embodiment 8: Inhibitory Effect of the Antibiotic Teicoplanin with Different Concentrations in Human Umbilical Vein Endothelial Cells (HUVECs)

(1) Packaging virus: plasmids pHIV-luc, pCMV-deltaR8.2 and Zaire EBOV-GP2014 were transfected into 293T cells (10 cm dish), and after 48 hours, a supernatant of virus was collected for testing p24.

Plasmids pHIV-luc, pCMV-deltaR8.2 and VSV-G were transfected into 293T cells (10 cm dish) at the same time, and after 48 hours, the supernatant of virus was collected for testing p24.

(2) Infecting: human umbilical vein endothelial cells (HUVECs) in a 96-well plate were infected by p24-normalized HIV-luc/Zaire EBOV-GP2014 or HIV-luc/VSV-G pseudotype virus containing 8 μg/mL polybrene, and the antibiotic teicoplanin with different concentrations were added at the same time.

(3) Changing medium: after 12 hours of infection, the medium was changed with fresh DNEM medium.

(4) Testing luciferase activity: after 48 hours of infection, each well was washed once with PBS, then 100 μL lysis buffer was added, a shaking was carried out for 30 min, and 10 μL of lysate was taken out for testing luciferase activity.

It can be observed from FIG. 8 that teicoplanin has good inhibitory effect on virus in the human umbilical vein endothelial cells (HUVECs).

1-5. (canceled)

6. A use of teicoplanin against Ebola virus.

7. The use of teicoplanin against Ebola virus according to claim 6, wherein the teicoplanin inhibits the envelope protein GP of Ebola virus.

8. The use of teicoplanin against Ebola virus according to claim 6, the envelope protein GP is Zaire type envelope protein of the Ebola virus that outbroke in 2014.

9. The use of teicoplanin against Ebola virus according to claim 6, wherein the teicoplanin inhibits the Ebola virus from entering into a host cell.

10. A drug inhibiting an envelope protein GP, wherein the drug comprises teicoplanin.
